(19) United States
(12) Patent Application Publication
(10) Pub. No.: US 2009/0082780 A1
(43) Pub. Date: Mar. 26, 2009

(54) WIRE CAPTURE SURGICAL DEVICE WITH FIXABLE HANDLE

(75) Inventors: Wenfeng Lu, Pfafftown, NC (US); Brian K. Rucker, King, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc, Winston-Salem, NC (US)

(21) Appl. No.: 12/211,591

(22) Filed: Sep. 16, 2008

Related U.S. Application Data
(60) Provisional application No. 60/975,298, filed on Sep. 26, 2007.

(51) Int. Cl. A61B 17/221; A61B 5/107

(52) U.S. Cl. 606/127; 600/587

(57) ABSTRACT

A medical device including a wire capture member such as, for example, a lithotripsy basket or a snare member, as well as a motion limiting means configured to limit a maximum expanded diameter of the wire capture member.

### CROSS-REFERENCE TO RELATED APPLICATIONS

This application claims priority to U.S. Provisional Application Ser. No. 60/975,298, filed Sep. 26, 2007, which is incorporated herein by reference in its entirety.

### FIELD OF THE INVENTION

The present invention relates to surgical devices, and more particularly to handle improvements for surgical wire capture devices, such as lithotripsy basket devices, and methods for the same.

### BACKGROUND

The gall bladder is an organ that stores bile secreted by the liver. The cystic duct from the gall bladder merges with the common hepatic duct, forming the common bile duct. A number of medical conditions are associated with various disorders, diseases, and injuries associated with the bile duct.

Choledocholithiasis is a medical condition associated with the entry of a biliary calculus (bile stone) into the bile duct. Obstruction of the bile duct can be excruciatingly painful for a patient suffering therefrom, and can cause nausea, fever, vomiting, and jaundice. Complete, persistent obstruction of the common bile duct can cause cholangitis, a life threatening infection of the biliary tree, which is a medical emergency. An obstruction of the common bile duct can also lead to an obstruction of the pancreatic duct, which may cause pancreatitis.

Several methods of treatment are used to remove the gall bladder and stones, including open surgery or laparoscopic surgery. Less invasive treatments may be used as well. For example, the stones may be removed endoscopically, without having to create any external incisions (e.g., by endoscopic cholelithotomy). In this technique, an endoscope is directed through the patient's esophagus to a location adjacent the Sphincter of Oddi, where the bile duct opens into the duodenum. Typically, a sphincterotome is used to cannulate and widen the sphincter opening to ease access into the bile duct for stone retrieval. A device including a basket deployable from a lumen of a catheter shaft may then be directed into the bile duct to capture stones for removal.

In some instances the stones are too large to pass through even the widened Sphincter of Oddi. If more invasive surgical techniques are to be avoided, then the stone must be crushed or broken into smaller pieces for removal (lithotripsy). A number of devices are known in the art for breaking up the stones. One such device is a mechanical lithotriptor basket device 100 comprising a wire basket 104 mounted on the distal end of an elongate basket wire 102, which is guided through a shaft 110 on a handle 112 to a location such that the basket 104 can be directed around a stone 106 (See FIGS. 1A-1C). Once the basket 104 is around the stone 106, the basket 104 is retracted toward and into the shaft 110, such that its internal volume is reduced. The compressive force caused thereby breaks or crushes the stone 106 into smaller pieces (See FIG. 1D) so that it can be removed or allowed to pass.

Endoscopic and laparoscopic snares are also well-known in the art. Snare devices operate very similarly to lithotriptors, except that they often have only a single wire loop rather than a multi-wire basket. A sample snare device 150 is illustrated with reference to FIG. 2, and includes a handle 152 attached to a shaft 154 and configured to actuate a drive wire 156. A snare loop 160 is disposed at the distal end of the drive wire 156. Snare devices such as the snare 150 may be used for resection or excision of tissues (such as, for example, polyps in the colon). Within the present application, snares are included in the concept of surgical basket devices generally and lithotriptors specifically, where either of those terms is used with reference to the present invention.

With current lithotriptors, snares, and other wire capture devices, there are numerous circumstances where it would be advantageous or even necessary to provide a limited diameter for the basket (when fully expanded) and/or a metric for determining the diameter of a structure being encompassed by the basket. For example, when using a prior art lithotriptor, it may be possible to capture—but not release—a stone that is too large to crush and remove. If that stone cannot be released, the patient being treated usually has to undergo an open surgical procedure to remove the stone and the engaged lithotriptor. This may also occur with so-called “peach stones” that have a softer exterior and a hard, pit-like core. If such a stone cannot be crushed or otherwise disrupted, the softer exterior material may capture the basket, necessitating removal by open surgical means. As another example, during an endoscopic cholelithotomy, it may be important to ascertain the maximum diameter of the cannulated sphincter of Oddi, as well as the diameter of a gallstone or fragment thereof in need of removal, so that one may determine whether the stone or fragment will safely be able to be withdrawn through the cannulated sphincter.

### BRIEF SUMMARY

Embodiments of the present invention may meet the aforementioned and other needs. For example, in one aspect, embodiments of the present invention may include a medical device including a wire capture member such as, for example, a lithotripsy basket or a snare member, and may also include a limiting means configured to limit a maximum expanded diameter of the wire capture member.

### BRIEF DESCRIPTION OF THE DRAWINGS

FIGS. 1A-1D show a prior art lithotripsy device and method of use thereof;

FIG. 2 shows a prior art snare device;

FIG. 3. illustrates an embodiment of a lithotriptor device of the present invention;

FIG. 4 depicts another embodiment of a lithotriptor device of the present invention;

FIG. 5 shows an embodiment of a snare device of the present invention; and

FIG. 6 shows a detail view of one handle embodiment of the present invention.

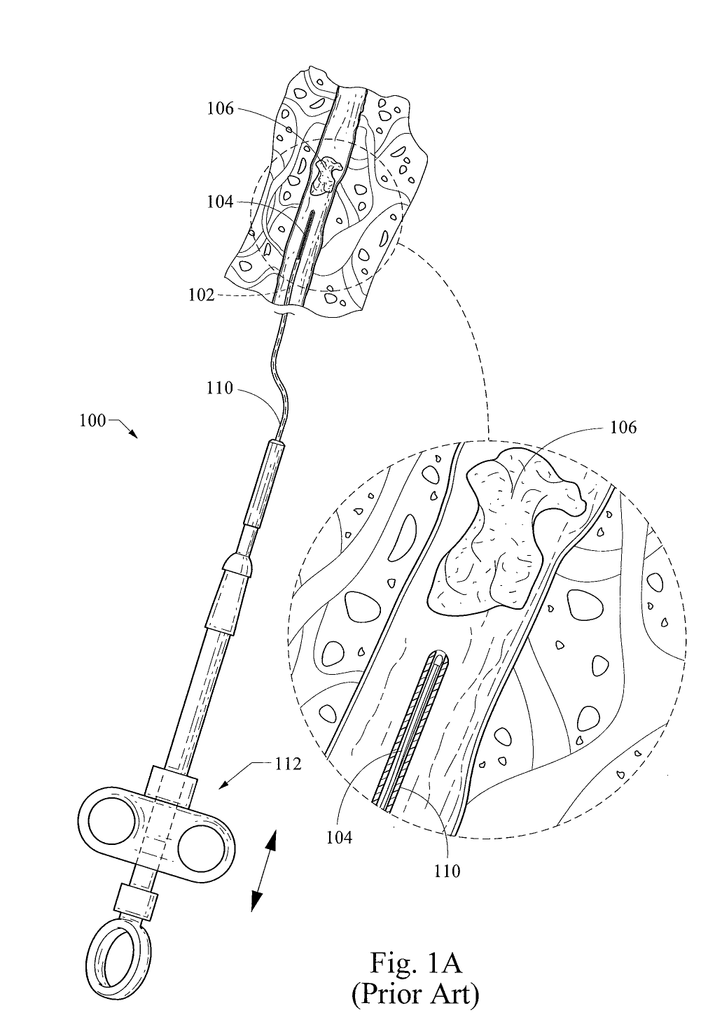

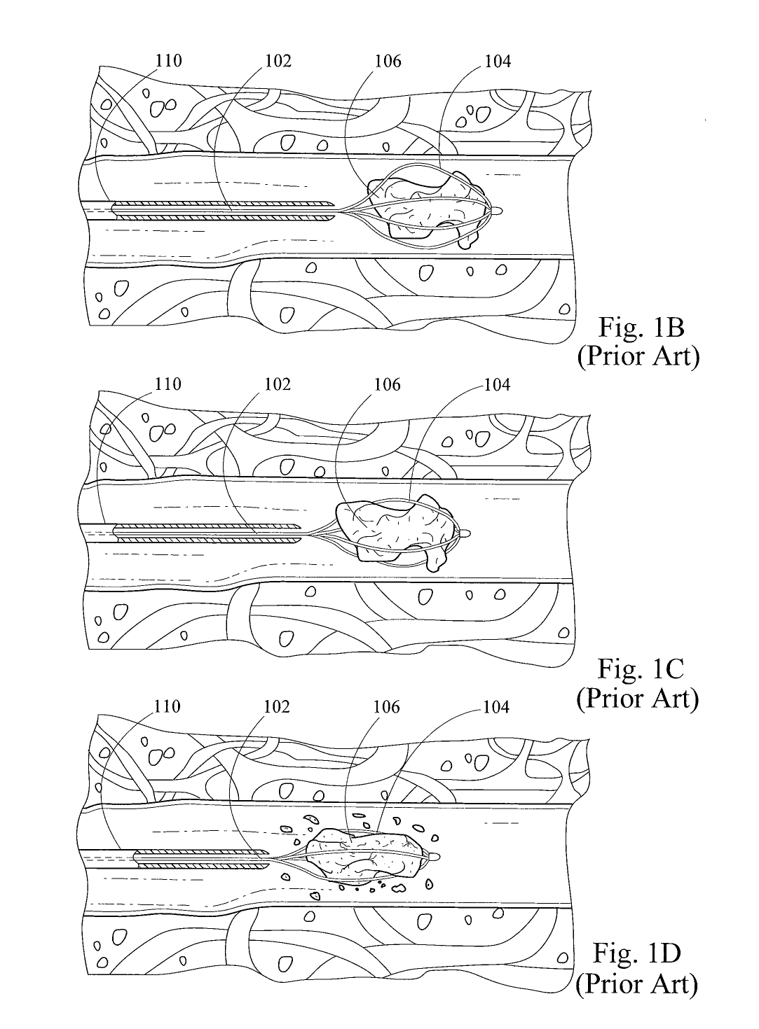

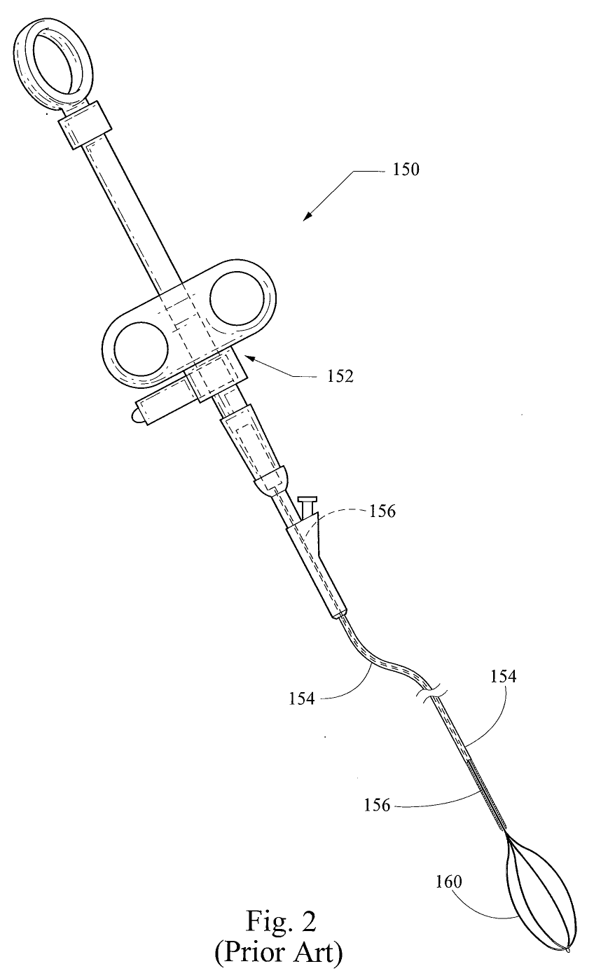

### DETAILED DESCRIPTION OF THE DRAWINGS AND THE PRESENTLY PREFERRED EMBODIMENTS

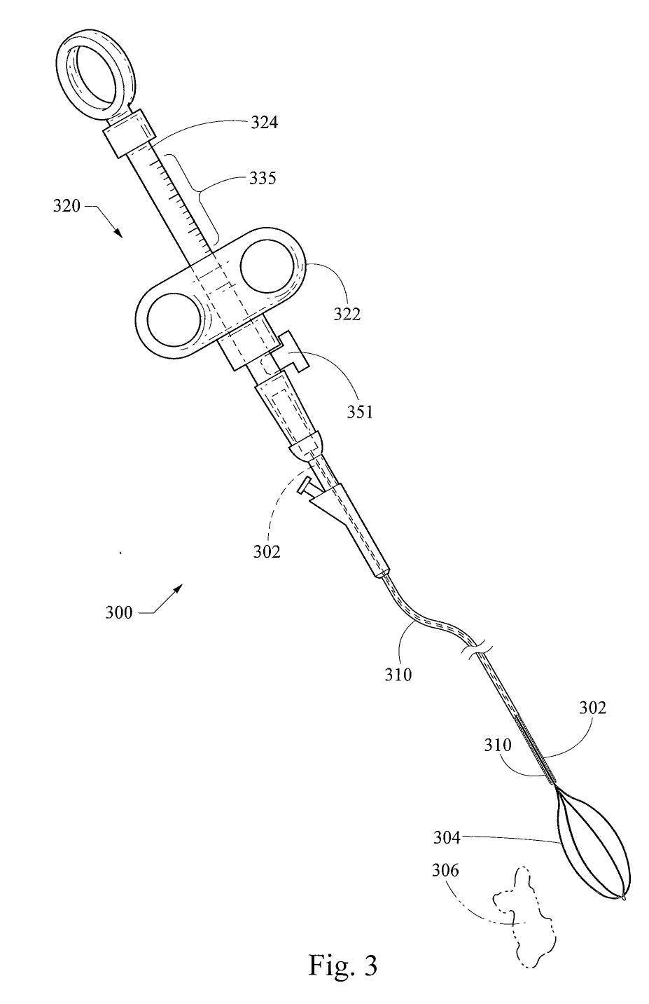

In one embodiment, illustrated with reference to FIG. 3, the present invention may include a surgical device embodied as a lithotripsy basket device 300 comprising a wire capture member embodied as a basket 304 mounted on the distal end of an elongate basket wire 302, which is slidably disposed through an elongate shaft 310 to a location such that the basket 304 can be directed around an object 306. The object 306 may be, for example, a gallstone, a foreign object in the alimentary canal, or another object elsewhere in a mammalian body (such as, for example, in the peritoneal cavity), and embodiments of the present invention are preferably configured for use through an endoscope within the peritoneal cavity (such as, for example, during an ERCP or Natural Orifice Translumenal Endoscopic Surgery (NOTES) procedure) whether within lumens communicating with the alimentary canal or outside of same.

The device 300 may include a handle 320, which is depicted here as a two-ring handle, but which those of skill in the art will appreciate may be embodied with other handle means appropriate for use with a lithotriptor. The two-ring handle 320 depicted includes a spool portion 322 that is slidably disposed about a stem portion 324. The stem portion 324 is attached to the shaft 310, and the spool portion 322 is attached to the basket wire 302. A groove 324a on a surface of the stem portion 324 engages a tongue member 322a of the spool portion 322 in a manner that allows a relative longitudinal sliding motion between the spool and stem portions 322, 324. (See FIG. 6, which shows a perspective view of handle actuation). Drawing the spool 322 proximally along the stem 324 or moving the stem 324 distally through the spool 322 will direct the shaft 310 and the basket 304 closer together, including extending the shaft 310 around the basket 304 (and/or drawing the basket into the shaft). Likewise, movement in the opposite direction will extend the basket 304 distally relative to the shaft 310 (and/or draw the shaft proximally relative to the basket). Those of skill in the art will appreciate that, in another embodiment, the shaft may be attached to the spool member and the drive wire attached to the stem member.

During an operation of the device, the basket 304 may be directed around the object 306. In a preferred embodiment, the wires forming the basket 304 are biased or otherwise formed such that, when extended out of the shaft 310, they spread apart to form a generally contained volume with a known diameter (the diameter preferably measured at the widest portion of the basket along a line crossing the longitudinal axis of the basket. Then the basket 304 may be retracted toward and into the shaft 310, such that its internal volume will be reduced to grasp the object 306. With the object 306 securely captured, it may be moved to another location to be released for elimination. Alternatively, or in addition, sufficient proximal force may be exerted on the basket wire 304 so as to compress the basket 304 and break or crush the object 306 into smaller pieces so that it can be removed (e.g., if in the alimentary canal, allowed to pass naturally through bodily elimination).

As shown in FIG. 3, the stem portion 324 includes marking indicia 335 that are shown in millimeters for illustration purposes. The position of the spool 322 relative to the indicia 335 corresponds to the expanded diameter of the basket 304 when it is extended distally from the shaft 310. The indicia 335 can be used for measuring the diameter of an object 306. For example, the basket 304 may be directed to and around an object 306, then drawn closely about it. A user may then observe the indicia to measure the diameter of the object 306. In a preferred embodiment of a method of measuring outer diameter, the object will include a generally regular outer diameter to be engaged by the basket such that measurement will be accurate, but those of skill in the art will appreciate that approximate measurement of the diameter of an irregularly-shaped object may also be valuable.

Similarly, the device 300 may be used for measuring the diameter of an opening such as, for example, a naturally occurring body orifice or passage portion, a surgically-created opening, or a surgically-enhanced body opening (e.g., the sphincter of Oddi, a pancreatic duct, a cannulated sphincter). Such measurement may be made by directing the basket 304 into a generally longitudinally centered position in the location to be measured, then actuating the handle portions 322, 324 to expand the basket 304 to contact the border of the region being measured, and observing the indicia 335 to determine a diameter of that region.

The device 300 may include a means for incrementally selectably limiting longitudinal motion of the spool 322 relative to the stem 324. Such means may be useful for limiting the diameter of an object 306 that can be captured by the basket 304 by limiting the extent to which the basket 304 may be extended from the shaft 310. This may be useful, for example, during an endoscopic gallstone removal or NOTES procedure where an object to be captured is too large to be withdrawn through the available bodily aperture. By limiting the “capture diameter” of the basket through the limiting means, an embodiment of the present invention may lessen the risk of capturing an object in the basket that is too large to be removed through an existing opening without causing injury and/or that may become trapped in the basket, necessitating an invasive surgical procedure.

For example, FIG. 3 shows the device 300 including a removable tap member 351 engaged into the groove 324a of the handle's stem portion 324. The location of the tap 351 distally from the spool 322 limits the distalward motion of the spool 322 such that the basket 304 cannot be fully extended from the shaft 310 and the maximum open diameter and capacity of the basket are limited. In a preferred system of the present invention, several taps of different sizes will be provided so that a user may select an appropriately sized tap to limit the effective size of the lithotriptor basket to a desired dimension. The taps preferably are color coded and/or otherwise marked with some form of indicia indicating the basket diameter to which each will limit the device 300.

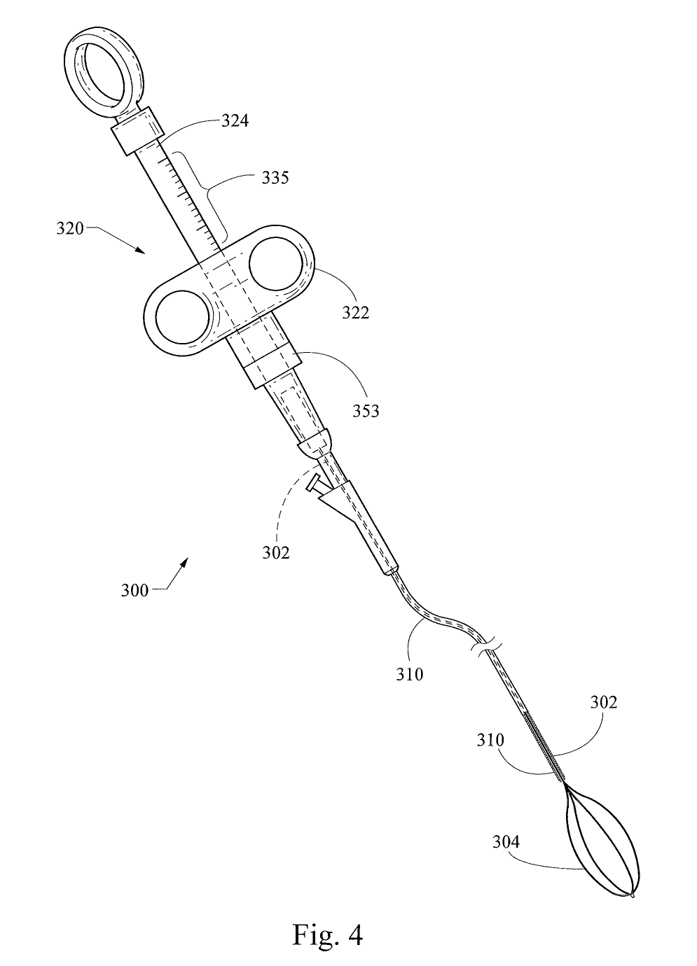

As another example, FIG. 4 shows the device 300 including a removable locking ring member 353 engaged around the handle's stem portion 324. The width of the ring 353 around the stem 324 limits the distalward motion of the spool 322 such that the basket 304 cannot be fully extended from the shaft 310 and the maximum open diameter and capacity of the basket are limited. In a preferred system of the present invention, several rings of different sizes will be provided so that a user may select an appropriately sized ring to limit the effective size of the lithotriptor basket to a desired dimension. The rings preferably are color coded and/or otherwise marked with some form of indicia indicating the basket diameter to which each will limit the device 300. Those of skill in the art will appreciate that numerous other means may be used to limit the relative motion of the handle portions including, for example, detent means, one or more adjustable set screws or flanges, and/or other motion-limiting means that will be apparent to those of skill in the art.

A method of use for a device 300 including a motion-limiting means (such as, for example, a tap or a locking ring) may include the following steps. A user wishing to remove an object may measure the diameter of an opening through which the object will have to pass, using the method described above. The user then may select a motion-limiting means that will limit the handle motion and thereby the effective diameter of the fully extended basket to a diameter that is about the same as, or preferably at least slightly less than that of the opening. The user may then proceed to direct the distal portion of the device 300 to a location adjacent the object and extend/open the basket to engage the object. If the object is of an appropriate size for the basket to engage, the user may capture the object in the basket and remove it. If the object is too large, the user may pursue other options (e.g., enlarge the opening, attempt removal of the object by other means).

The device may also include means for enhancing mechanical advantage in retracting/constricting the basket such as, for example, the handle means disclosed in U.S. patent application Ser. No. 11/765,009 to Kennedy, which is incorporated by reference herein in its entirety.

In one example of a method of the present invention during an ERCP procedure, a lithotripsy device of the present invention may be directed through a working channel of an endoscope to a cannulated Sphincter of Oddi. The handle may be actuated to extend the basket distally out of the shaft such that about a longitudinal midpoint of the basket is generally aligned with the cannulated sphincter. The basket may be opened to contact the borders of the sphincter in a manner expanding the sphincter to its widest non-injurious diameter. A user may observe the indicia on the handle to measure the spread diameter of the sphincter. A motion-limiting means such as, for example, a tap or a locking ring, may be placed upon the handle to limit the diameter of an object (e.g., a gallstone) that may be captured by the basket such that the basket will have a decreased likelihood of capturing an object too large to be drawn through the sphincter if that object cannot be crushed by a standard lithotripsy operation of the basket. Then, the basket may be directed adjacent an object to be captured. If the object is too large to be captured, the basket won't engage that object, and the cannulated sphincter may need to be widened, or the object may need to be removed by other means. If the object is of a size that is appropriate to be captured, the basket may be engaged around it. In one embodiment of the method, the user may measure the stone by drawing the basket snugly around the object and observing the indicia on the handle to observe the diameter of the stone (e.g., for diagnostic or other recordal purposes). Then the object may be crushed by a standard lithotripsy operation of the basket, or it may be withdrawn intact and released in the duodenum.

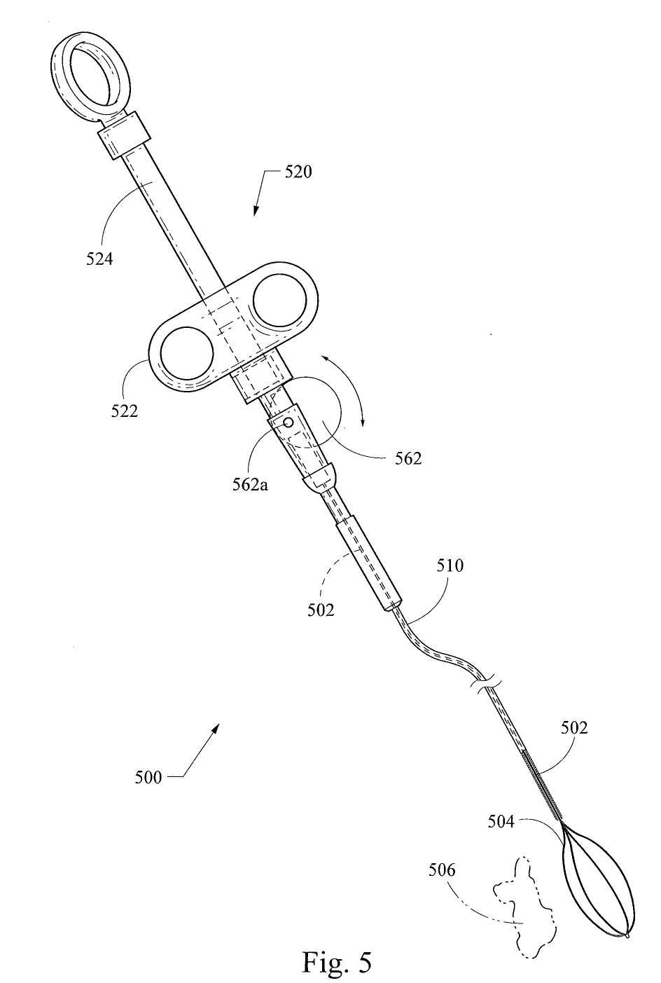

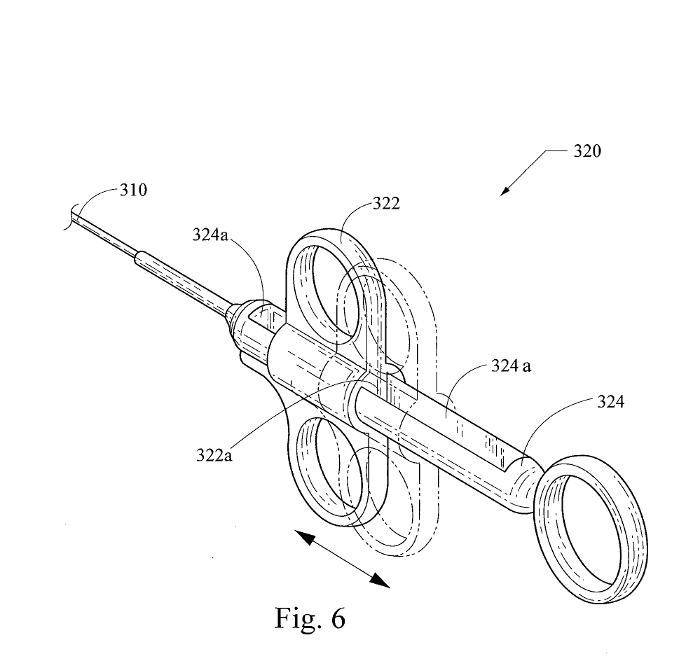

In one embodiment, illustrated with reference to FIG. 5, the present invention may include a surgical device embodied as a surgical snare 500 comprising a wire capture member embodied as a loop member 504 mounted on the distal end of an elongate drive wire 502, which is slidably disposed through an elongate shaft 510 to a location such that the loop member 504 can be directed around an object 506. The object 506 may be, for example, tissue mass such as a polyp, a foreign object in the alimentary canal, or another object elsewhere in a mammalian body (such as, for example, in the peritoneal cavity), and embodiments of the present invention are preferably configured for use through an endoscope within the peritoneal cavity (such as, for example, during an ERCP or Natural Orifice Translumenal Endoscopic Surgery (NOTES) procedure) whether within lumens communicating with the alimentary canal or outside of same.

The snare 500 may include a handle 520, which is depicted here as a two-ring handle, but which those of skill in the art will appreciate may be embodied with other handle means appropriate for use with a snare. The two-ring handle 520 depicted includes a spool portion 522 that is slidably disposed about a stem portion 524. The stem portion 524 is attached to the shaft 510, and the spool portion 522 is attached to the drive wire 502. In the same manner as is illustrated with reference to FIG. 6, a groove on a surface of the stem portion engages a tongue member of the spool portion in a manner that allows a relative longitudinal sliding motion between the spool and stem portions 522, 524. Drawing the spool 522 proximally along the stem 524 or moving the stem 524 distally through the spool 522 will direct the shaft 510 and the loop member 504 closer together, including extending the shaft 510 around the loop member 504 (and/or drawing the basket into the shaft). Likewise, movement in the opposite direction will extend the loop member 504 distally relative to the shaft 510 (and/or draw the shaft proximally relative to the basket). Those of skill in the art will appreciate that, in another embodiment, the shaft may be attached to the spool member and the drive wire attached to the stem member.

The snare 500 may include a motion-limiting means of the type described above with reference to FIGS. 3-4, or another type. FIGS. 5A-4B illustrate the snare 500 with a motion-limiting means embodied as an offset locking wheel 562. The offset locking wheel 562 may include a generally circular or other shaped member mounted to rotate about an off-center axis 562a in a position configured to limit the relative motion of the stem 524 and spool 522 in a manner that restricts the maximum extended diameter of the loop member 504. Those of skill in the art that this wheel means may be used with lithotriptor embodiments or other embodiments within the scope of the present invention. The locking means for the wheel 562 is not shown explicitly, but those of skill in the art will appreciate that it may be embodied as a locking detent or one of many other known wheel-locking means known in the art.

The offset mounting of the wheel 562 allows it to be rotated to restrict movement of the stem 524 relative to the spool 522. Those of skill in the art will appreciate that rotation of the wheel 562 in a first direction (e.g., counterclockwise in FIG. 5) will decrease the relative movement potential of the handle members 522, 524 and thereby reduce the maximum loop diameter.

Those of skill in the art will also appreciate that numerous variations of the invention described above may be practiced within the scope of the present invention. It is therefore intended that the foregoing detailed description be regarded as illustrative rather than limiting, and that it be understood that it is the following claims, including all equivalents, that are intended to define the spirit and scope of this invention.

We claim:

1. A lithotripsy basket device comprising:
a handle;
a shaft having a longitudinal lumen and extending distally from the handle;
a drive wire extending through the shaft lumen; and
a lithotripsy basket disposed adjacent a distal end of the drive wire, said lithotripsy basket comprising a plurality of wires configured to expand generally radially apart from each other to at least a first predetermined diameter;
wherein said handle comprises a first handle member is slidably disposed relative to a second member and is connected to the drive wire, being configured such that actuation of one handle member relative to the other moves the drive wire longitudinally relative to the shaft;
and wherein said handle further comprises a means for incrementally selectably limiting longitudinal motion of the first handle member relative to the second handle member such that the radial expansion of the plurality of wires is correspondingly limited.

2. The lithotripsy basket device of claim 1, further comprising marking indicia upon one of the first and second handle members, said marking indicia configured to correspond to a diameter measurement of a lithotripsy basket portion extending beyond a distal end of the shaft.

3. The lithotripsy basket device of claim 2, wherein the marking indicia correspond to the first predetermined diameter.

4. The lithotripsy basket device of claim 2, wherein the marking indicia correspond to a second predetermined diameter.

5. The lithotripsy basket device of claim 1, wherein the means for limiting longitudinal motion comprises at least one removable tap member disposed between a portion of the first handle member and a portion of the second handle member.

6. The lithotripsy basket device of claim 1, wherein the means for limiting longitudinal motion comprises at least one locking clamp member attached to one of the first and second handle members and configured to limit a motion thereof relative to the other of the first and second handle members.

7. A method for measuring the diameter of an object, said method comprising the steps of:
providing the lithotripsy basket device of claim 2;
directing the basket such that it encompasses an object;
actuating the handle such that the basket closely engages the object; and
observing the marking indicia to determine the diameter of the basket as closely engaged around the object.

8. The method of claim 7, wherein the object is a gallstone.

9. The method of claim 7, wherein the object is located within the peritoneal cavity of a mammal.

10. A method for measuring the diameter of an opening, said method comprising the steps of:
providing the lithotripsy basket device of claim 2;
directing the basket such that it is generally longitudinally centered in an area generally transverse to a plane defined by an opening;
actuating the handle such that the basket opens and closely engages a circumference defined by the opening; and
observing the marking indicia to determine the diameter of the opening as defined by the diameter of the open basket.

11. The method of claim 10, wherein the opening is a naturally-occurring orifice in the body of a mammal.

12. The method of claim 10, wherein the opening is a surgically-created opening in the body of a mammal.

13. The method of claim 10, wherein the opening is a surgically-enlarged, naturally-occurring orifice in the body of a mammal.

14. A surgical device comprising:
a handle;
a shaft having a longitudinal lumen and extending distally from the handle;
a drive wire extending through the shaft lumen; and
a wire capture member disposed adjacent a distal end of the drive wire, said wire capture member comprising at least one wire loop configured to expand generally radially open to at least a first predetermined diameter;
wherein said handle comprises a first handle member is slidably disposed relative to a second member and is connected to the drive wire, being configured such that actuation of one handle member relative to the other moves the drive wire longitudinally relative to the shaft;
and wherein said handle further comprises a means for incrementally selectably limiting longitudinal motion of the first handle member relative to the second handle member such that the radial expansion of the plurality of wires is correspondingly limited.

15. The surgical device of claim 14, further comprising marking indicia upon one of the first and second handle members, said marking indicia configured to correspond to a diameter measurement of a wire capture member portion extending beyond a distal end of the shaft.

16. The surgical device of claim 15, wherein the marking indicia correspond to the first predetermined diameter.

17. The surgical device of claim 14, wherein the means for limiting longitudinal motion comprises at least one removable tap member disposed between a portion of the first handle member and a portion of the second handle member.

18. The surgical device of claim 14, wherein the means for limiting longitudinal motion comprises at least one locking clamp member attached to one of the first and second handle members and configured to limit a motion thereof relative to the other of the first and second handle members.

19. The surgical device of claim 14, wherein the means for limiting longitudinal motion comprises an offset wheel member attached to one of the first and second handle members and configured to limit a motion thereof relative to the other of the first and second handle members.

20. The surgical device of claim 14, wherein the wire capture member is selected from a snare loop and a lithotripsy basket.
